(19) United States
(12) Patent Application Publication
(10) Pub. No.: US 2024/0184367 A1
(43) Pub. Date: Jun. 6, 2024

(54) SENSORY FEEDBACK SYSTEM

(71) Applicant: BIONIC HOPE PRIVATE LIMITED, Thane (IN)

(72) Inventors: Llewellyn GRENOLD LOY DSA, Thane (IN); Priyanka DSA, Thane (IN); Anil NAIR, Thane (IN)

(21) Appl. No.: 18/279,396

(22) PCT Filed: Mar. 1, 2022
(86) PCT No.: PCT/IN2022/050172

§ 371 Date: Aug. 30, 2023

(30) Foreign Application Priority Data
Mar. 1, 2021 (IN) 202121008628

(51) Int. Cl. G06F 3/01; A61B 34/00; A61B 34/35; B25J 13/02; B60Q 9/00; B64D 45/00; G08B 6/00

(52) U.S. Cl. CPC G06F 3/016; A61B 34/76; B25J 13/025; B60Q 9/00; B64D 45/00; A61B 34/35; G08B 6/00

(57) ABSTRACT

The present disclosure relates to a sensory feedback system (101) including a sensor array (104) and a sensory feedback processing unit (106). The sensor array (104) is arranged with respect to an entity (102) to sense a set of parameters associated with an operation of the entity (102). The sensory feedback processing unit (106) is configured to generate sensory feedback based on the sensed parameters, and provide the sensory feedback to an operator of the entity (102) by way of at least one of: an actuator and an output device, thereby enabling control of the operation of the entity (102). The sensory feedback is at least one of: tactile feedback, visual feedback, and audio feedback.

### FIELD OF THE INVENTION

The present invention relates to a sensory feedback system, and more specifically to a system for providing sensory feedback for better control and operation of one or more automated devices.

### BACKGROUND

With the advancement in the technology, people are ever more reliant on automated devices for their day-to-day activities. Several devices have been developed which help people with doing different tasks. Such devices either use a pre-programmed code or rely on human intervention to finish their daily tasks.

Further, some devices have also been developed which make use of sensors to collate information from the sensor and use them for their functioning. For example, a driver of a car when reversing has limited vision and has to rely on rear view cameras or rear-view sensors to make the necessary decisions, as cameras and existing sensors have blind spots. However, the driver still has difficulties during parallel parking, reversing into cramped areas and driving in traffic because the driver cannot feel the vehicles environment. The rear view cameras and rear view sensors only provide a view or sense of a limited area to the driver, thereby not providing much avoidance of any mishappening in orientations where blind spots exist.

Furthermore, the devices using sensors can sense information in one line of path in a limited area and based on only the external sensors being used. Such devices do not consider one or more inputs or one or more outputs provided by various internal components of the device. Hence, the information provided by such devices is not accurate for all orientations of sensing. Also, such devices do not help a new person operating the device in any way. Every time a new person has to use the device, the person has to be trained first before actually using the device. This makes the whole process cumbersome in cases where the one or more automated devices are used by different persons working in different shifts or when a new person has to handle an automated device for the first time in case of some emergency.

Conventionally, Light Detection and Ranging (LiDAR) sensors are used on the devices for sensing information. LiDAR sensors are a type of laser distance sensor that measure the range, or depth from a surface. They work by emitting pulses in all directions and measuring how long it takes for them to bounce back off targets. The working principle of LiDAR sensors is similar to that of ultrasonic sensors. The only difference is the frequency in which they operate, while LiDARs use a laser beam instead of sound waves for measuring distance and analysing objects with laser beams generated from an array or cluster.

LiDAR sensors have the ability to measure 3D structures and generally aren't affected by light. They have a large measurement range and very good accuracy. Small objects are generally detected well with LiDAR sensors as they have smaller wavelengths than sonar sensors. If you're trying to detect something moving quick, the fast update rate will allow you to detect those targets as well.

The limitations of using LiDAR include a higher cost as compared to ultrasonic and infrared (IR) sensors. It is also harmful for the naked eye as high end LiDAR devices may use stronger pulses that could affect human eyes, which means it must have a safety guard installed on top of each sensor in order not be damaged by sunlight exposure or bright light reflections off water surfaces. Narrow point detection can miss some objects like glass and items in close proximity to the floor.

Thus, in view of the above, there is a need to provide a system that provides sensory feedback for better control and operation of one or more automated devices. As sensory stimulation taps into the wide array of tactile memory available to any given user, reducing the learning curve. Further, there is a need of a new sensor array to overcome the shortcomings of LiDAR.

### SUMMARY OF THE INVENTION

This summary is provided to introduce a selection of concepts, in a simple manner, which are further described in detailed description of the invention. This summary is neither intended to identify the key or essential inventive concept of the subject matter, nor to determine the scope of the invention.

According to one aspect of the present disclosure, there is provided a sensory feedback system including a sensor array and a sensory feedback processing unit. The sensor array is arranged with respect to an entity to sense a set of parameters associated with an operation of the entity. The sensory feedback processing unit configured to generate sensory feedback based on the sensed parameters, and provide the sensory feedback to an operator of the entity by way of at least one of: an actuator and an output device, thereby enabling control of the operation of the entity. The sensory feedback is at least one of: tactile feedback, visual feedback, and audio feedback.

Additionally, or optionally, the entity is a vehicle and the operation of the entity includes navigation along a route. The sensor array includes a plurality of proximity sensors arranged with respect to the vehicle in one or more orientations of the vehicle to cover one or more blind spots of the vehicle. The set of parameters include at least one of: a distance between the vehicle and one or more obstacles in the one or more orientations, and a shape of the one or more obstacles. The sensory feedback processing unit provides the sensory feedback to an operator of the vehicle in respect to the control of the navigation of the vehicle, through at least one of: a seat of the operator, a steering mechanism of the vehicle, a control pedal of the vehicle, and a display device of the vehicle. The tactile feedback is at least one of: heat, pressure, displacement, vibration, electrical, and texture based simulation.

Additionally, or optionally, the entity is a movable arm of an equipment having a pivoting element and the operation of the entity includes handling of the entity. The sensor array includes a plurality of motor current sensors arranged to sense a torque at a pivoting point of the pivoting element. The set of parameters include a force applied by the movable arm. The sensory feedback processing unit provides the sensory feedback to an operator of the equipment in respect to the control of the entity and the load carried by the entity, through at least one of: a seat of the operator, a steering mechanism of the equipment, a remote control device of the equipment, and a display device of the equipment. The tactile feedback is at least one of: heat, pressure, displacement, vibration, electrical, and texture based simulation.

Additionally, or optionally, the equipment is one of: an industrial vehicle, an industrial robot, a medical equipment, a prosthetic device, and a bomb defusal equipment.

Additionally, or optionally, the entity is a movable arm of an equipment having a gripping element and the operation of the entity includes handling load carried by the entity. The sensor array includes a plurality of sensors arranged to sense imbalance or tilt at a gripping point of the gripping element. Each sensor is at least one of: a proximity sensor and a pressure sensor. The set of parameters include a back pressure at the movable arm and an orientation of load with respect to the movable arm. The sensory feedback processing unit provides the sensory feedback to an operator of the equipment in respect to the control of the load held by the entity, through at least one of: a seat of the operator, a steering mechanism of the equipment, a remote control device of the equipment, and a display device of the equipment. The tactile feedback is at least one of: heat, pressure, displacement, vibration, electrical, and texture based simulation.

Additionally, or optionally, the equipment is one of: an industrial vehicle, an industrial robot, a medical equipment, a prosthetic device, and a bomb defusal equipment.

Additionally, or optionally, the entity is an unmanned aerial vehicle and the operation of the entity includes handling orientation and navigation of the entity. The sensor array includes a plurality of sensors arranged with respect to the unmanned aerial vehicle in one or more orientations of the unmanned aerial vehicle to cover one or more blind spots of the unmanned aerial vehicle, and maintain orientation of the unmanned aerial vehicle. Each sensor is at least one of: a proximity sensor and an orientation sensor. The set of parameters includes at least one: of a proximity of one or more obstacles with respect to the entity, and pitch, yaw, and roll of the entity. The sensory feedback processing unit provides the sensory feedback to an operator of the equipment in respect to the control of the orientation and navigation of the entity, through a remote control device of the unmanned aerial vehicle. The tactile feedback is at least one of: heat, pressure, displacement, vibration, electrical, and texture based simulation.

Additionally, or optionally, the entity is one or more wearable bands, and the operation of the entity includes assistive guidance to a person having visual impairment. The sensor array includes a plurality of proximity sensors arranged on the one or more wearable bands to sense the set of parameters associated with assistive guidance to the person regarding obstructions in front of the person. The set of parameters includes at least one of: a distance between the person and one or more obstacles and a shape of the one or more obstacles. The sensory feedback processing unit provides the tactile feedback to the person in respect to the control of assistive guidance provided to the person, through the one or more wearable bands. The tactile feedback is at least one of: heat, pressure, displacement, vibration, electrical, and texture based simulation.

Additionally, or optionally, the entity is a wearable spectacle, and the operation of the entity includes assistive guidance to a person having at least one of: visual impairment and hearing impairment. The sensor array includes a plurality of proximity sensors arranged on the wearable spectacle to sense the set of parameters associated with assistive guidance to the person regarding obstructions in front or side of the person. The set of parameters includes at least one of: a distance between the person and one or more obstacles and a shape of the one or more obstacles. The sensory feedback processing unit provides the tactile feedback to the person in respect to the control of assistive guidance provided to the person, through one or more temple tips of the wearable spectacles. The tactile feedback is at least one of: heat, pressure, displacement, vibration, electrical, and texture based simulation.

Additionally, or optionally, the entity is one of: a wearable band and an exoskeleton worn on a body part of a patient, and the operation of the entity includes monitoring of muscle health of the body part. The sensor array includes a plurality of sensors arranged on one of: the wearable band and the exoskeleton to sense the set of parameters associated with muscle health of the body part. The sensory feedback processing unit provides the tactile feedback to the patient in respect to the monitoring of muscle health of the body part and the visual and/or audio feedback to a healthcare professional monitoring the patient, through one or more actuators of one of: the wearable band and the exoskeleton. The tactile feedback is at least one of: heat, temperature, pressure, texture, vibro-tactile, electro-tactile, and mechano-tactile based simulation.

The sensory feedback system of the present disclosure includes a sensor array arranged with respect to an entity in one or more orientations of the entity to cover one or more blind spots of the entity and provide improved control over the operations of the entity such as navigation. Thus, avoiding any mishappening due to the one or more blind spots. Further, based on the sensed information the sensory feedback processing unit provides sensory feedback (tactile or visual or audio feedback) to an operator of the entity to provide more efficient control over operations of the entity and also may be able to train a new person operating the entity. Furthermore, the present sensory feedback system utilizes sensors such as ultrasonic, optical, motor current, pressure, proximity, orientation, and the like that lave lower cost than LiDAR, and are also not harmful for the naked eye. As a result, the sensory feedback system helps in controlling various operations in applications such as utility, industrial, robotic, assistive technology, medical, and the like.

Further benefits, goals and features of the present invention will be described by the following specification of the attached figures, in which components of the invention are exemplarily illustrated. Components of the devices and method according to the inventions, which match at least essentially with respect to their function, can be marked with the same reference sign, wherein such components do not have to be marked or described in all figures.

The invention is just exemplarily described with respect to the attached figures in the following.

### BRIEF DESCRIPTION OF DRAWINGS

The invention will be described and explained with additional specificity and detail with the accompanying figures in which:

FIG. 1 illustrates an environment, wherein various embodiments of the present invention can be practiced;

FIGS. 2A-2C illustrate exemplary sensor arrays;

FIGS. 3A and 3B illustrate various view of two configurations of sensor arrays;

FIGS. 4A-4D illustrate use of one or more sensor arrays with a vehicle, in accordance with an embodiment of the present disclosure;

FIG. 4E illustrates exemplary tactile feedback provided to a driver of the vehicle;

FIGS. 4F and 4G illustrate exemplary visual feedback provided to the driver of the vehicle;

FIGS. 5A-5C illustrate implementation of a sensory feedback processing unit in an industrial vehicle, in accordance with another embodiment of the present disclosure;

FIGS. 6A-6D illustrate implementation of a sensory feedback processing unit in medical field, in accordance with yet another embodiment of the present disclosure;

FIG. 7 illustrates an exemplary application of the sensory feedback processing unit, where a controlled robot is used for bomb defusal process;

FIGS. 8A and 8B illustrate another exemplary application of the sensory feedback processing unit, where a medical equipment is controlled remotely by a healthcare worker;

FIG. 9 illustrates yet another exemplary application of the sensory feedback processing unit, where a drone is used in a search and rescue operation;

FIGS. 10A and 10B illustrate another exemplary application of the sensory feedback processing unit, where the sensory feedback system is used as an assistive device for blind and deaf people;

FIG. 11 illustrates another application of the sensory feedback processing unit, where the sensory feedback system is used in diagnosing and treating muscle atrophy; and

FIG. 12 illustrates another application of the sensory feedback processing unit, used along with a hand exoskeleton in administering physiotherapy to prevent muscle atrophy.

Furthermore, the figures may show only those specific details that are pertinent to understanding the embodiments of the present invention so as not to obscure the figures with details that will be readily apparent to those skilled in the art having the benefit of the description herein.

### DETAILED DESCRIPTION OF INVENTION

For the purpose of promoting an understanding of the principles of the invention, reference will now be made to the embodiment illustrated in the figures and specific language will be used to describe them. It will nevertheless be understood that no limitation of the scope of the invention is thereby intended. Such alterations and further modifications in the illustrated system, and such further applications of the principles of the invention as would normally occur to those skilled in the art are to be construed as being within the scope of the present invention.

It will be understood by those skilled in the art that the foregoing general description and the following detailed description are exemplary and explanatory of the invention and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this invention belongs. The system, methods, and examples provided herein are only illustrative and not intended to be limiting.

Embodiments of the present invention will be described below in detail with reference to the accompanying figures.

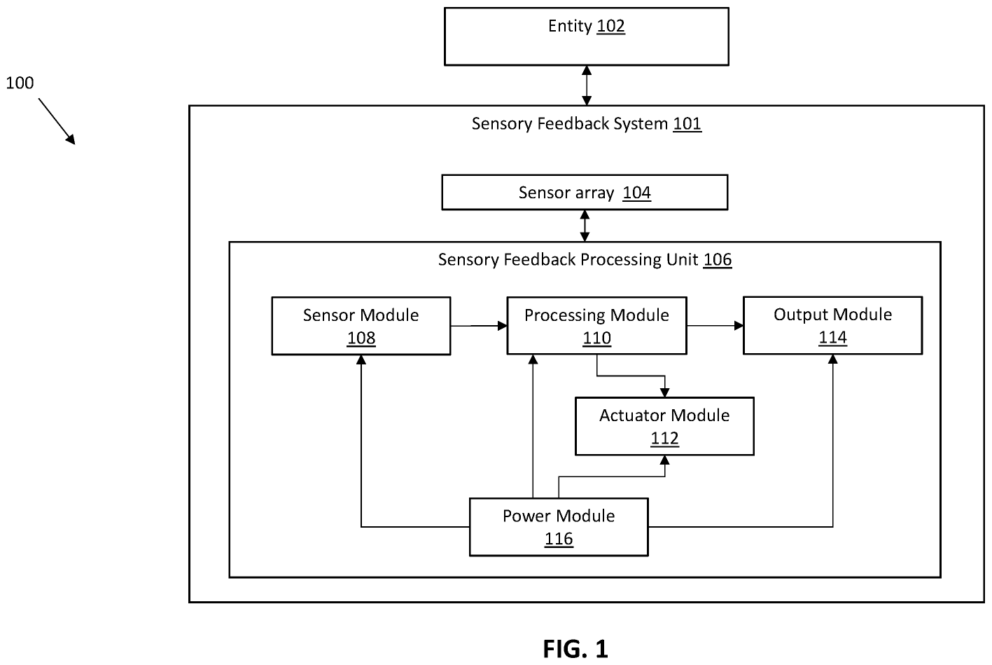

FIG. 1 illustrates an environment 100, wherein various embodiments of the present invention can be practiced. The environment 100 includes an entity 102, and a sensory feedback system 101 in communication with the entity 102. The entity 102 performs and controls a set of operations. The entity 102 may be utilized in public/private utility applications, industrial applications, robotic applications, assistive technology applications, medical applications, or the like. Examples of the entity 102 include, but not restricted to, a human, a robot, any other automated device, an industrial vehicle, a mobile phone, a laptop, a tabloid, any other device with a user interface, medical equipment, etc. Examples of the set of operations include, but are not limited to, navigation, pivoting or gripping, assembling, mounting, controlling orientation, and assistive guidance.

It will be understood by a person skilled in the art that although in the current embodiment, only one entity is shown, in various other embodiments, any number of entities may be in communication with the sensory feedback system 101, without deviating from the scope of the present disclosure. The multiple entities may be communicatively coupled to each other through a communication network. The communication network may be any suitable wired network, wireless network, a combination of these or any other conventional network, without limiting the scope of the present disclosure. Few examples may include a Local Area Network (LAN), wireless LAN connection, an Internet connection, a point-to-point connection, or other network connection and combinations thereof.

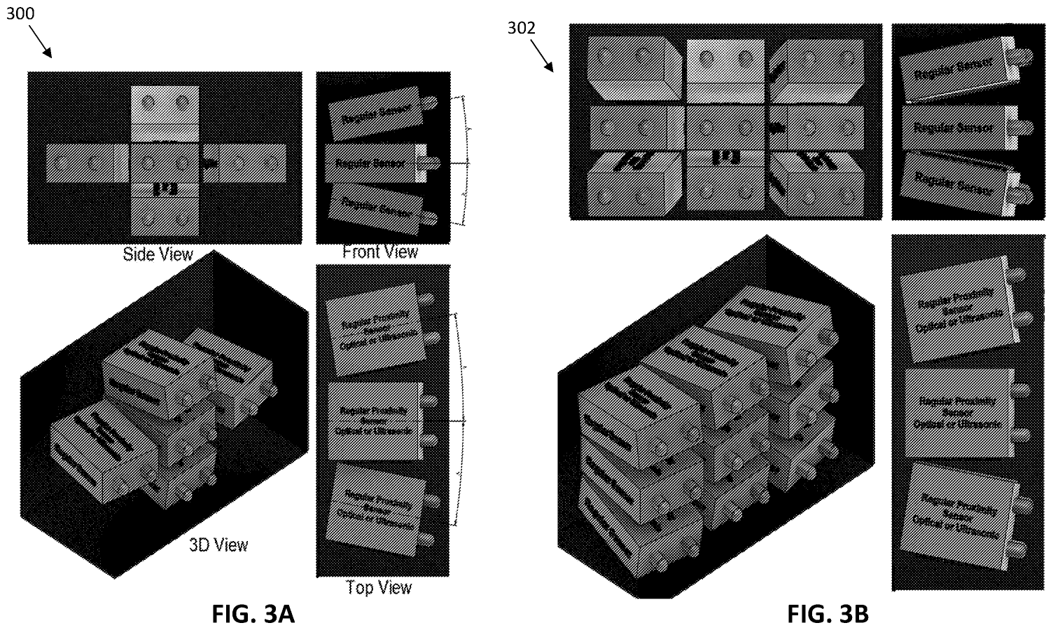

The sensory feedback system 101 includes a sensor array 104 and a sensory feedback processing unit 106. The sensor array 104 is arranged with respect to the entity 102 to sense a set of parameters associated with an operation of the entity 102, and generate a sensor signal based on the sensed set of parameters. Each sensor array includes a plurality of sensors (shown later in FIGS. 3A and 3B). The sensor array 104 may include, but not limited to, one type of sensor or a combination of different type of sensors. Further, types of sensors may include, but not limited to, resistive, capacitive, pressure sensitive, bio-potential, electrical, mechanical, optical, barometric etc. In one embodiment, the examples of the plurality of sensors include, but are not limited to, a proximity sensor, an orientation sensor, a motor current sensor, and a pressure sensor.

The sensory feedback processing unit 106 helps in controlling and operating the entity 102 in an efficient manner. The sensory feedback processing unit 106 generates sensor data based on the sensor signal, processes the sensor data to generate sensory feedback based on the processed sensor data, and provides the sensory feedback to an operator of the entity 102 by way of at least one of: an actuator and an output device, thereby enabling control of the operation of the entity 102. The sensory feedback is at least one of: tactile feedback, visual feedback, and audio feedback.

It will be understood by a person skilled in the art that although in the current embodiment, only one sensor array and one sensory feedback processing unit is shown, in various other embodiments, any number of sensor arrays and sensory feedback processing units may be included in the sensory feedback system 101, without deviating from the scope of the present disclosure. The multiple sensory feedback processing unit may be communicatively coupled to each other through the communication network.

The sensory feedback processing unit 106 includes a sensor module 108, a processing module 110, an actuator module 112, an output module 114, and a power module 116. The sensor module 108 is coupled with the sensor array 104, and receives the sensor signals from the sensor array 104 attached to the entity 102 (externally or internally) and generates sensor data based on the sensor signal. The sensor module 108 provides the sensor data to the processing module 110.

The processing module 110 is coupled with the sensor module 108, and receives the sensor data from the sensor module 108 and processes the sensor data to generate processed sensor data (hereinafter also referred to as “processed information”). Based on the processed information and one or more simulations supported by the entity 102, the processing module 110 provides the processed information to the actuator module 112, or the output module 114, or to both.

If the entity 102 supports one or more tactile simulations, then the processing module 110 sends the processed information to the actuator module 112. If the entity 102 supports one or more display devices, then the processing module 110 sends the processed information to the output module 114. Further, if the entity 102 supports both tactile simulations and display devices, then the processing module 110 sends the processed information to both the actuator module 112 and the output module 114.

The actuator module 112 is coupled with the processing module 110, and receives the processed information and based on the processed information provides tactile feedback associated with the control of the operation of the entity 102 to an operator of the entity 102 by way of an actuator of the actuator module 112. The tactile feedback includes, but not limited to, feedback in the form of heat, pressure, displacement, electrical, vibration, and texture based simulation or change. The actuator converts the processed information into corresponding heat, pressure, displacement, electrical, vibration, or texture based simulation. Examples of the one or more actuators include, but are not limited to, electric, pneumatic, or hydraulic actuators.

The output module 114 is coupled with the processing module 110 to receive the processed information, and provide at least one of: the audio feedback and the visual feedback associated with the control of the operation of the entity 102 to the operator of the entity 102 by way of an output device of the output module 114. The output device is at least one of: an audio equipment and a visual equipment. Examples of the audio equipment include, but are not limited to, a speaker, a headset, a buzzer, and a headphone/earphone. Examples of the visual equipment include, but are not limited to, a display of corresponding entity, a display of a user device of the user, a display of wearable device of the user, and indicator lights. The power module 116 provides power for proper functioning of the sensor module 108, the processing module 110, the actuator module 112, and the output module 114.

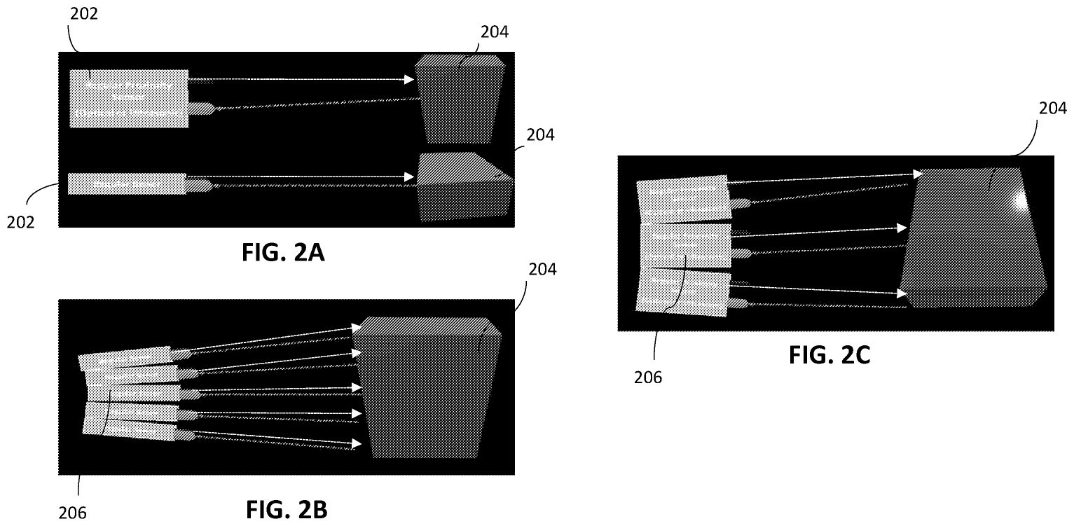

FIGS. 2A-2C illustrate exemplary sensor arrays. FIG. 2A illustrates top view and front view of a sensor 202, such as a regular proximity sensor based on optical signal or ultrasonic signal. The sensor 202 transmits a signal, such as an optical or ultrasonic signal, towards an obstacle 204 and receives the signal reflected from the obstacle 204 to determine a parameter, such as a proximity of the obstacle 204 from the entity 102 on which the sensor 202 is attached. FIG. 2B illustrates front view of the proximity sensors used to map shapes of obstacles. FIG. 2C illustrates top view of regular proximity sensors used to map shapes of the obstacles. Five rows and three columns of proximity sensors similar to the sensor 202 are used in FIGS. 2B and 2C to map an obstacle, such as the obstacle 204.

FIGS. 3A and 3B illustrate various view of two configurations of sensor arrays 300 and 302. FIG. 3A illustrates side, front, top, and perspective view of a configuration of sensor array 300. As shown in FIG. 3A, the sensor array 300 includes five sensors such that a first sensor is in centre and the remaining fours sensors are on top, bottom, left, and right of the first sensor with each sensor having a predefined angular displacement with respect to adjacent sensor. FIG. 3B illustrates side, front, top, and perspective view of another configuration of sensor array 302. As shown in FIG. 3B, the sensor array 302 includes nine sensors such that there are three rows and three columns of sensors with each sensor having the predefined angular displacement with respect to adjacent sensor. Thus, due to arrangement of sensor based on above configuration enables the sensors to cover maximum area around the entity 102. As a result, when the one or more sensor arrays 300 or 302 are coupled with the entity 102 all orientations for sensing are covered, thereby covering one or more blind spots of the entity 102.

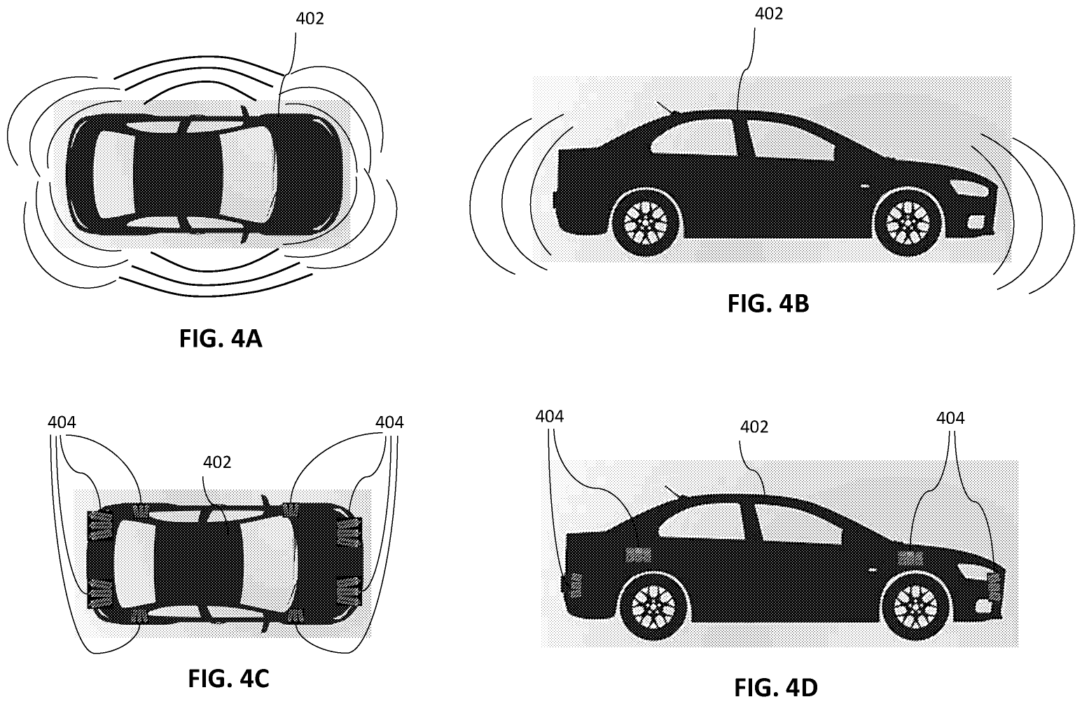

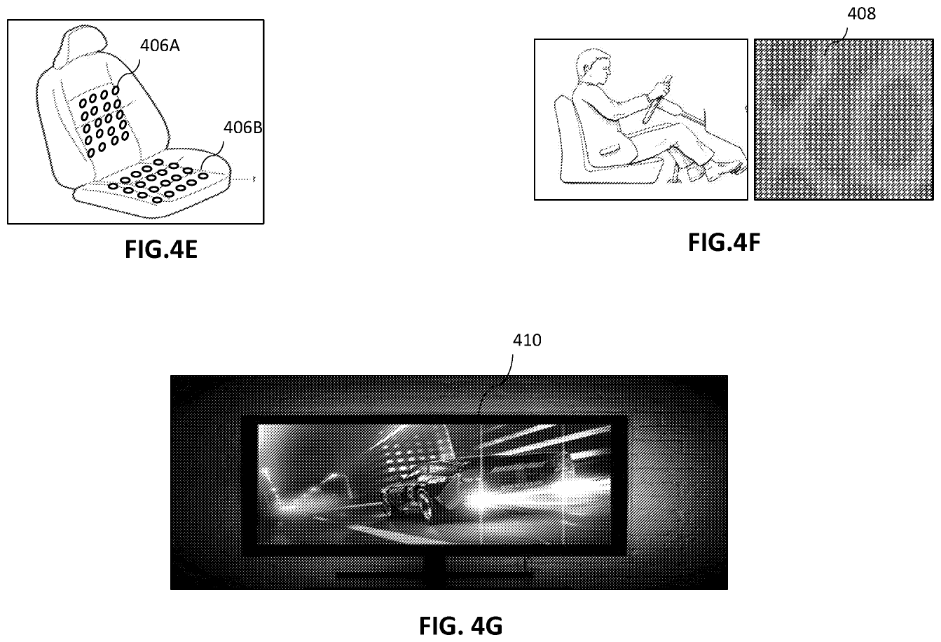

FIGS. 4A-4D illustrate implementation of the sensory feedback processing unit 106 and the one or more sensor arrays 404 with a vehicle 402, in accordance with an embodiment of the present disclosure. In this embodiment, the entity 102 is the vehicle 402 and the operation of the entity includes navigation along a route, such as a route between source location and destination location. In FIGS. 4A and 4C, a top view of the vehicle 402 is shown. Multiple sensor arrays 404 are arranged with respect to the vehicle 402 to sense the set of parameters in all orientations of the vehicle 402 to cover one or more blind spots of the vehicle 402. In one example, the set of parameters includes a distance between the vehicle 402 and obstacles in a surrounding region (in one or more orientations) of the vehicle, and a shape of all the obstacle. Based on the sensed set of parameters, the sensor arrays 404 provide information about proximity of obstacles in all the directions around the vehicle 402 to the sensory feedback processing unit 106 communicatively coupled to the sensor arrays 404. In one embodiment, each sensor array 404 includes a plurality of proximity sensors, and has a configuration similar to the configuration of sensor array described in FIG. 3B (i.e., nine proximity sensors arranged in three rows and three columns). In another embodiment, each sensor array 404 includes a plurality of proximity sensors, and has a configuration similar to the configuration of sensor array described in FIG. 3A (i.e., five proximity sensors arranged such that four sensors are on top, bottom, left and right of a sensor in centre).

In FIGS. 4B and 4D, a side view of the vehicle 402 is shown. The one or more sensor arrays 404 scan and provide information about proximity of obstacles along a height of the vehicle 402 to the sensory feedback processing unit 106. The sensory feedback processing unit 106 is either internal or external to the vehicle 402. In this embodiment, the proximity sensor is implemented by way of an optical sensor or an ultrasonic sensor. The sensory feedback processing unit 106 provides the sensory feedback (i.e., at least one of: the tactile feedback, the visual feedback, and the audio feedback) in respect to the control of the navigation of the vehicle 402, through at least one of: a seat of the operator or user, a steering mechanism of the vehicle 402, a control pedal of the vehicle 402, and a display of the vehicle 402. In one embodiment, the tactile feedback is at least one of: heat, pressure, displacement, vibration, electrical, and texture based simulation. In one example, the display device of the vehicle 402 is capable of providing audio output along with video output.

FIG. 4E illustrates exemplary tactile feedback provided to a driver (or operator) of the vehicle 402. The tactile feedback includes tactile simulations 406A and 406B embedded into a seat of a driver of the vehicle 402. The tactile simulations 406A and 406B may be one of heat, pressure, displacement, vibration, electrical, or texture based stimulations, or a combination of them. Based on the processed information received from the processing module 110, the actuator module 112 actuates the tactile simulations 406A and 406B, thereby alerting the driver to avoid any mishappening. The tactile simulations 406A and 406B may include, but not limited to, vibration simulation, electric simulation, rapid heating on the side of proximity from the obstacle, change in texture, or a combination thereof. Further, the tactile simulations 406A and 406B may be present on other parts of the vehicle 402 as well. For example, the steering wheel, gloves of the driver, control pedals, control panels, etc., individually or in combination. In one embodiment, the tactile simulations 406A and 406B are actuated by way of one or more actuators (not shown).

Further, the output module 114 receives the processed information and provides visual feedback or audio feedback to the operator of the entity 102. The visual feedback includes, but not limited to, a colour matrix-based display highlighting any obstacles around a device, a backlighting-based system to highlight any obstacles around a device (where colour and intensity of the light varies with proximity of the obstacles from the device), graph display of the processed information on a user device, or any other types of display supported by the user device. The audio feedback includes, but not limited to, an audio system (such as connected speakers) of the vehicle 402 may provide audio alerts, a wireless audio device of the user that is connected to the vehicle 402, and a buzzer, where volume and tone of the audio varies with proximity of the obstacles from the device.

FIGS. 4F and 4G illustrate exemplary visual feedback provided to the driver on a display of the vehicle 402. FIG. 4F illustrates a colour matrix 408 displayed on a visual screen, which is a two-dimensional display array with RGB colour mapping, of the vehicle 402, where the colour matrix 408 displays proximity of one or more obstacles from the vehicle 402. FIG. 4G illustrates change in backlighting 410 of a rear-view camera or a display screen of the vehicle 402 to signify obstacles, where colour and intensity of the backlighting 410 varies with proximity of the obstacles from the vehicle 402.

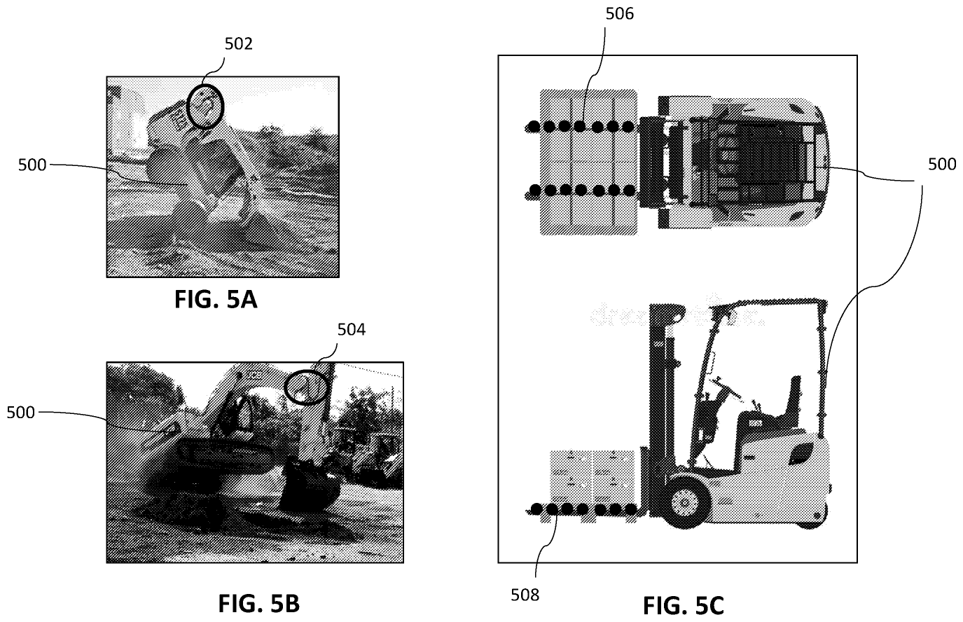

FIGS. 5A-5C illustrate implementation of the sensory feedback processing unit 106 in industrial vehicle, in accordance with another embodiment of the present disclosure. In FIGS. 5A-5C, the entity 102 is a movable arm of an equipment 500 having a pivoting element or a gripping element. In one embodiment, the equipment is an industrial vehicle such as, a bobcat, a bull dozer, an excavator, a forklift, and the like. The sensory feedback processing unit 106 is used with one or more industrial vehicles. In case of industrial vehicles like bobcats, bull dozers, and forklifts, operators need to be trained well to operate such equipment as a certain degree of judgement is required to handle the vehicle and load properly. If too much force is applied by an arm of the vehicle or the load is not lifted in a well-balanced position, it can cause the vehicle to tip over and can lead to loss of life and property.

In FIGS. 5A-5C, an array of sensors (not shown) is mounted on the industrial vehicles at positions 502, 504, 506, and 508, to detect forces applied by the movable arms of the industrial vehicles. Further, force can also be measured by using back pressure in hydraulic systems of the industrial vehicles during loading. The sensor array may sense a set of parameters such as a force applied by the movable arm, a back pressure at the movable arm, and an orientation of load with respect to the movable arm. In one embodiment, the sensors at the positions 502 and 504, i.e., the pivot points of the movable arms, are motor current sensors that sense the torque at the pivot points 502 and 504 and the user may be alerted via the feedback mechanism in case of excessive torque that may cause damage to the equipment and the user of the equipment. Further, the array of sensors at the positions 506 and 508, i.e., the gripping or loading point of the arms, is a proximity sensor, or a pressure sensor, or a combination of both that sense the imbalance or tilt at the gripping or loading points 506 and 508 and the user is alerted via the feedback mechanism in case of improper loading that may cause damage to the equipment and the user of the equipment. The sensor module 108 receives the detected torque or load information, generates corresponding sensor data, and provides the sensor data to the processing module 110. The processing module 110 processes the sensor data to check for any imbalance or improper loading of the vehicle based on pressure, load, contact made by the arms of the vehicles and sends the processed information to the actuator module 112 or the output module 114 or both. Based on the information received, the actuator module 112 actuates one or more tactile simulations, thereby alerting the operator to avoid any mishappening. The tactile simulations may include, but not limited to, vibration simulation, electric simulation, rapid heating of the simulations based on amount of force, change in texture, or a combination thereof. Further, the tactile simulations may be present in different parts of the industrial vehicles. For example, operator seat, the steering wheel, gloves of the operator, control panels, etc.

Further, the output module 114 receives the processed information and provides visual feedback or audio feedback or both to the operator of the vehicle. The visual feedback includes, but not limited to, a colour matrix-based display highlighting imbalance or improper loading, a backlighting-based system to highlight force applied or imbalance or improper loading (where colour and intensity of the light varies with amount of force applied by the vehicle), a graph display of the processed information on a user device, or any other types of display supported by the user device. The audio feedback includes, but not limited to, an audio system (such as connected speakers) of the industrial vehicle may provide audio alerts, a wireless audio device of the user that is connected to the industrial vehicle, and a buzzer, where volume and tone of the audio varies with amount of force applied by the vehicle or the arms of the vehicle.

The tactile feedback and/or the audio-visual feedback provided by the sensory feedback processing unit 106 to the operator, helps to alert the operator, thereby avoiding any mishappening. Further, it also helps to control the industrial vehicle and the load carried by the industrial vehicle in an efficient manner, without causing any wear and tear to the vehicle. Also, it helps as a simulator for training new operators.

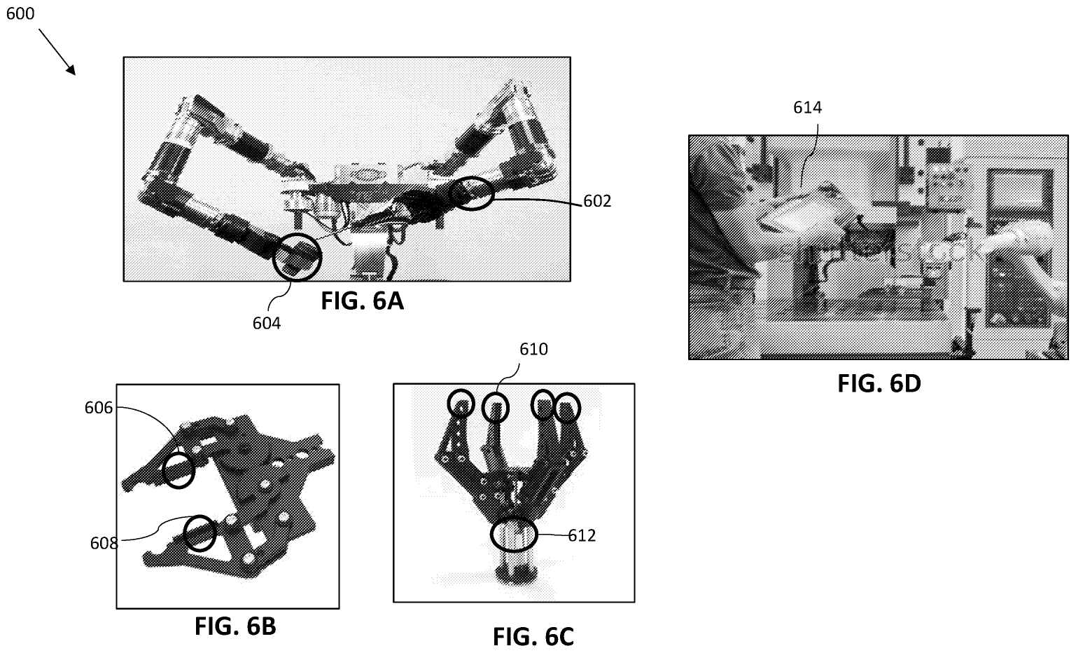

FIGS. 6A-6D illustrate implementation of the sensory feedback processing unit 106 in medical systems, in accordance with yet another embodiment of the present disclosure. In FIGS. 6A-6D, the entity 102 is a movable arm of an equipment 600 and the sensory feedback processing unit 106 sends tactile feedback and audio-visual feedback to the operator wirelessly controlling the equipment 600. In this embodiment, the equipment 600 is an industrial robot or a medical equipment or a prosthetic device. In FIG. 6A, sensor arrays are attached to joints 602 (pivot points) and fingertips 604 (gripping points) of the grippers of the equipment 600. The sensor array at the joints 602 includes motor current sensors that scan and provide information on back pressure on the jaws of the gripper and the sensor array at the fingertips 604 includes proximity or pressure sensors that provide information on the grip force of the jaws of the gripper. Similarly, in FIG. 6B, sensor arrays at the gripping point 606 and 608 include proximity or pressure sensors that scan and provide information on the grip force and contact made by the jaws of the gripper. Also, in FIG. 6C, sensor array at gripping point 610 includes proximity or pressure sensors that scan and provide information on the grip force and contact made by the jaws of the gripper and sensor array at pivoting point 612 includes motor current sensors that scan and provide information on the back pressure on the jaws of the gripper.

The sensor module 108 receives all the information from the sensor arrays at positions 602-612 by way of sensor signals and also receives back-current information from a motor controlling the gripper by way of the motor current sensor, and generates the sensor data. The sensor module 108 sends the sensor data to the processing module 110. The processing module 110 processes the sensor data to generate the processed information and provides the processed information to the actuator module 112 or the output module 114 or both. The actuator module 112 and the output module 114 provide tactile simulations and/or audio-visual output (as described in previous figures) to the user controlling the load held by the robot 600 on a user device 614.

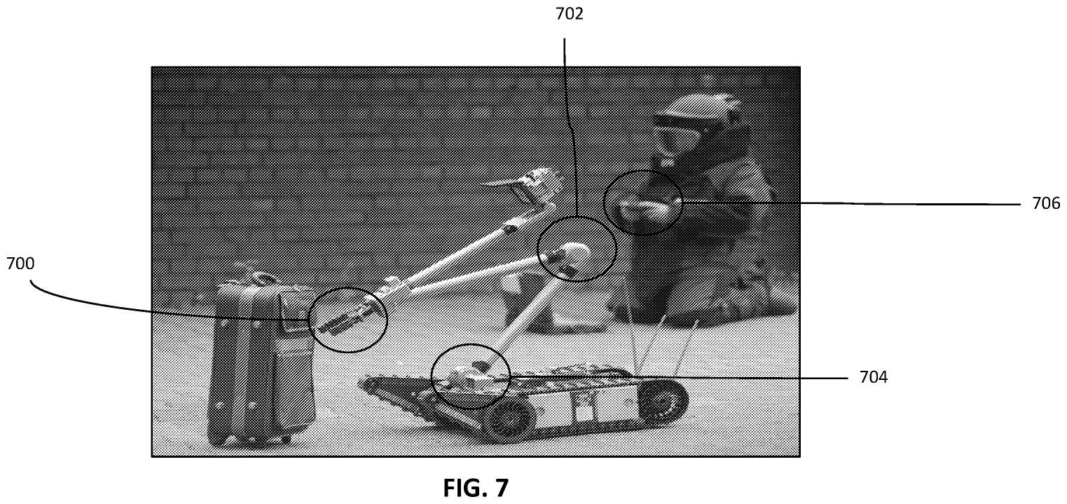

The embodiment, as described in FIGS. 6A-6D, can be used in multiple applications. FIG. 7 illustrates an exemplary application of the sensory feedback processing unit 106, where a controlled robot is used for bomb defusal. As explained in FIGS. 6A-6D, sensors attached to the robot provide information on grip force and contact made by jaws of a gripper of the robot. The sensors also provide information on back pressure on the jaws of the gripper and back current from a motor controlling the gripper. Based on the information provided by the sensors, the sensory feedback processing unit 106 provides feedback in the form of tactile simulations and/or visual output (as described in previous figures) to a user controlling the robot. Hence, the user can control the robot remotely and direct the robot in the defusal of the bomb.

As seen in FIG. 7, sensor arrays 700, 702, and 704 are attached to joints of the arm and fingertips of the grippers of the robot. The sensor arrays 702 and 704 include motor current sensors that scan and provide information on back pressure on the arm. In case of overloading or malfunction, an amount of torque increases and back pressure on the arm increases which can be alerted to the user by way of the feedback mechanism. The sensor array 700 includes proximity or pressure sensors that provide information on the grip force of the jaws of the gripper. In case of improper gripping or loading, the equipment may malfunction or the object to be gripped or loaded may fall leading to devastating effects in scenarios like bomb defusal, so such malfunction can be alerted to the user by way of the feedback mechanism. Each sensor array of the one or more sensor arrays 700, 702, and 704 includes a plurality of sensors that are arranged to sense the set of parameters associated with the one or more pivoting or gripping elements to monitor working of the robot. In one example, while performing in close proximity to a target object the sensor arrays 300 and 302 described in FIGS. 3A and 3B may be helpful in navigating the robot.

The sensor module 108 receives all the information from the sensors 700-706 by way of sensor signals and also receives back-current information from a motor controlling the gripper by way of the motor current sensor, and generates the sensor data. The sensor module 108 sends the sensor data to the processing module 110. The processing module 110 processes the sensor data to generate the processed information and provides the processed information to the actuator module 112 or the output module 114 or both. The actuator module 112 and the output module 114 provide tactile simulations and/or audio-visual output (as described in previous figures) to the user controlling the robot on a user device 706.

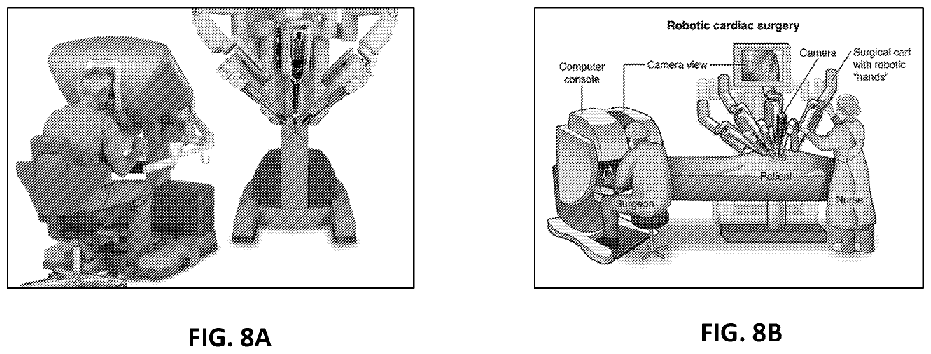

Further, FIGS. 8A-8B illustrate another exemplary application of the sensory feedback processing unit 106, where a medical equipment is controlled remotely by a healthcare worker. As explained in FIGS. 6A-6D, one or more sensors attached to a medical equipment provide information on grip force and contact made by jaws of a gripper of the medical equipment. The sensors also provide information on back pressure on the jaws of the gripper and back current from a motor controlling the gripper. Based on the information provided by the sensors, the sensory feedback processing unit 106 provides feedback in the form of tactile simulations and/or visual output (as described in previous figures) to the healthcare worker. Hence, the healthcare worker can remotely perform diagnosis on a patient and perform surgery on the patient remotely. In this embodiment, motor current sensors are implemented in the sensor arrays for joints, proximity sensors or pressure sensors, or a combination of both is implemented in the sensor arrays for gripping mechanism, pressure sensor is implemented in the sensor arrays for piercing mechanism, and a remote control mechanism along with a display is implemented to provide tactile or audio-visual feedback to the operator of the device. The tactile feedback would enable the surgeon to perform the surgery using this system much more efficiently as they would be able to feel the strength and stiffness of tissue while operating on the patient remotely.

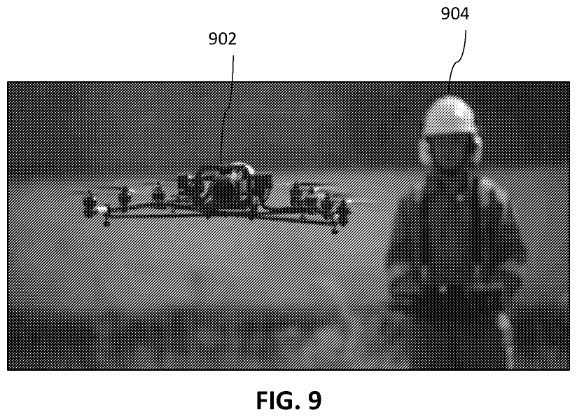

Furthermore, FIG. 9 illustrates yet another exemplary application of the sensory feedback processing unit 106, where a drone 902 (i.e., an unmanned aerial vehicle) is used in a search and rescue operation. In this embodiment, the entity is the drone 902 and the operation of the entity includes handling and orientation of the drone. The drones used in search and rescue operation and surveillance in tight spots need skill of a user to navigate. FIG. 9 illustrates the drone 902 being controlled by a user 904. The sensor array includes a plurality of sensors arranged with respect to the drone 902 in one or more orientations of the drone 902 to cover one or more blind spots of the unmanned aerial vehicle 902 and maintain orientation of the drone 902. In operation, the sensor array detects information of obstacles around the drone 902, and also pitch, yaw, and roll of the drone 902. The information of obstacles around the drone 902 may be detected using proximity sensors, and pitch, yaw, and roll of the drone 902 may be detected using orientation sensor that provide information regarding the inertial measurements related to the drone 902. Thus, the sensed set of parameters includes at least one of: a proximity of one or more obstacles with respect to the drone 902, and pitch, yaw, and roll of the drone 902. The sensory feedback processing unit 106 receives the detected information from the sensors and sends tactile feedback and/or audio-visual feedback (as explained in previous figures) to the user 904 by way of the remote control device used by the user 904 to control the drone 902, thereby providing the user 904 better results and helping the user 904 to control the navigation and orientation of the drone 902 in an efficient manner.

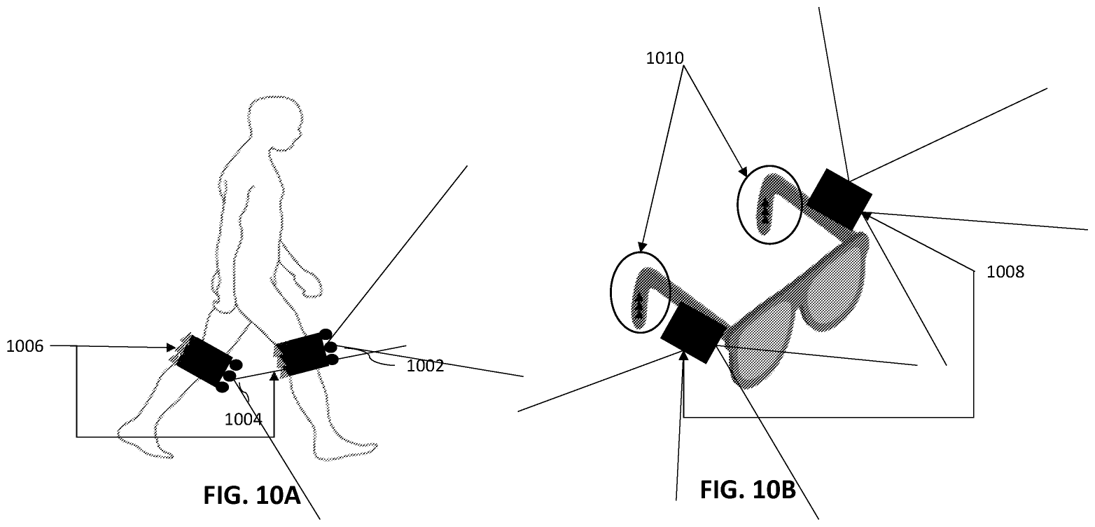

Further, FIG. 10A and FIG. 10B illustrate another exemplary application of the sensory feedback processing unit 106, where the sensory feedback processing unit 106 is used as an assistive device for people with visual and hearing impairment. FIG. 10A illustrates the use of the sensory feedback processing unit 106 for providing tactile simulations to a person having visual impairment. In FIG. 10A, an array of sensors 1002 and 1004 is placed inside wearable bands, which the person can wear around his/her knees. The sensor arrays 1002 and 1004 in wearable bands include a plurality of sensors which may be implemented as proximity sensors. The sensor arrays 1002 and 1004 inside the bands perform scanning to detect stairs as well as to identify objects in front of the person across the entire height of the person for providing assistive guidance to the person. The set of parameters includes at least one of: a distance between the person and one or more obstacles and a shape of the one or more obstacles The sensors send the detected information as sensor data to the sensory feedback processing unit 106. On receiving the detected information as the sensor data, the sensory feedback processing unit 106 provides tactile simulations to the person having visual impairment, through the one or more wearable bands, behind the knees at position 1006 to alert the visually impaired person about his or her surroundings. The advantage of using the sensory feedback system 101 in this application is that the sensory feedback system 101 alerts the person by scanning the area around the person in all directions, in contrast to conventional systems that provide feedback based on scanning performed in a single line of path. Hence, the sensory feedback system 101 of the present application helps the visually impaired person to take tougher paths such as stairs without falling and walk handsfree without using an assistive stick or similar device.

FIG. 10B illustrates the use of the sensory feedback processing unit 106 for providing tactile simulations to a person having visual and/or hearing impairment. People having hearing impairment find it difficult to sense oncoming traffic or obstacles from the side. Hence, FIG. 10B illustrates an array of sensors 1008 provided on the sides of wearable spectacles worn by the person. The set of parameters includes at least one of: a distance between the person and one or more obstacles and a shape of the one or more obstacles. Based on the information scanned by the sensors 1008, the sensory feedback processing unit 106 provides tactile simulations to the person having hearing impairment, through one or more temple tips of the wearable spectacles, behind the earlobes at position 1010, thereby timely alerting the person with visual and/or hearing impairment about the oncoming traffic.

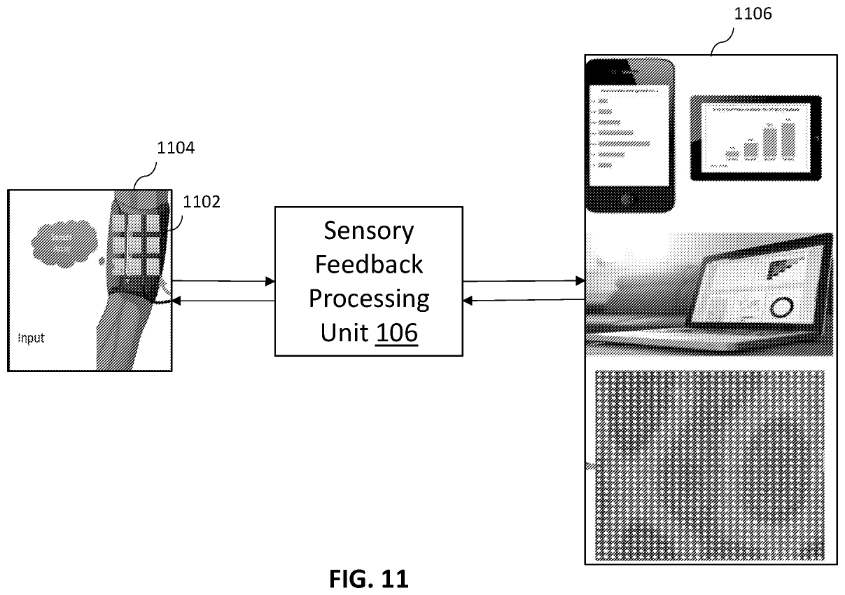

In yet another application, the sensory feedback system 101 is used to diagnose and treat muscle atrophy in case of a person with issues like, but not limited to, paralysis, nerve damage, cognitive damage, injury or amputation. Muscle atrophy is when muscles waste away. It's usually caused by a lack of physical activity. When a disease or injury makes it difficult or impossible for an individual to move an arm or leg, the lack of mobility can result in muscle wasting. FIG. 11 illustrates another application of the sensory feedback processing unit 106, used in diagnosing and treating muscle atrophy. In order to diagnose, a wearable band with inbuilt sensors and feedback actuators 1102 is wrapped around a body part 1104 of a patient. The sensor arrays include a plurality of sensors arranged to sense a set of parameters associated with the muscle health of the body part 1104 and send the information to the sensory feedback processing unit 106. The sensory feedback processing unit 106 processes the information and provides visual and/or audio feedback on the condition of the muscle to a healthcare professional on one or more output devices 1106, as well as if configured back to the wearable band. In order to treat muscle atrophy, the healthcare professional analyses the information displayed on the output devices 1106 (i.e., a display device) and sends a direction to the sensory feedback processing unit 106. The sensory feedback processing unit 106 provides tactile simulations to the body part 1104 of the patient, through the actuators 1102 of the wearable band, which can be controlled by the professional for amplitude, frequency, modulation and patterns. The tactile simulations may include, but not limited to, heat, pressure, vibro-tactile, electro-tactile, or mechano-tactile simulations. Further, in order to treat muscle atrophy, the healthcare professional can regulate various means like frequency, amplitude, etc., based on the readings observed from output devices 1106.

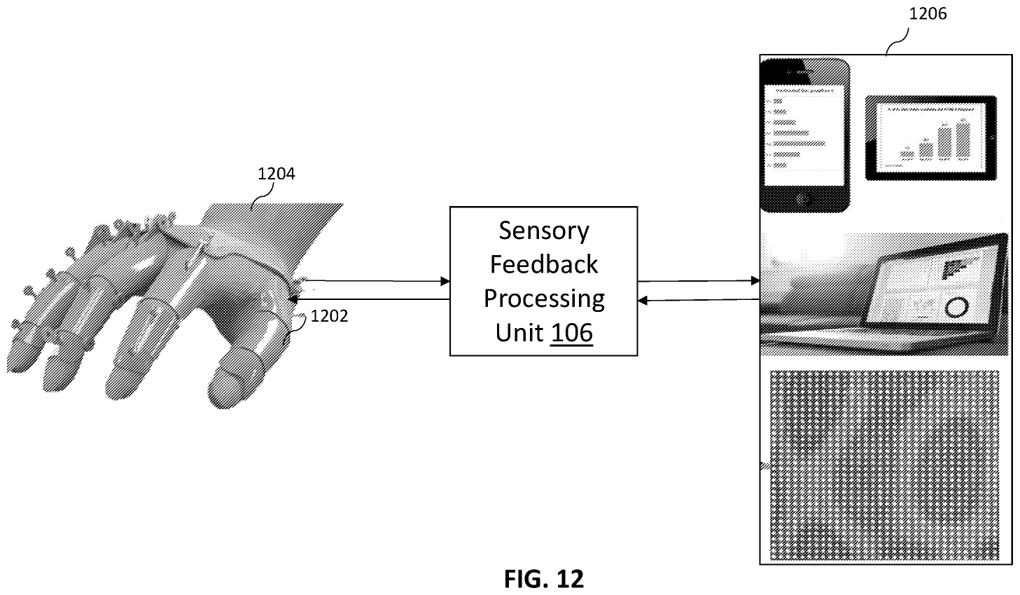

In yet another application, the sensory feedback system 101 coupled with an exoskeleton that is used to treat conditions like muscle atrophy, cerebral palsy, paralysis in the form of Mechanized Physiotherapy with Feedback in order to not just regain muscle movement as well as sensations like touch, pressure, temperature etc. When a disease or injury makes it difficult or impossible for an individual to move an arm or leg, the lack of mobility can result in muscle wasting or Muscle Atrophy, with Physiotherapy administered through an exoskeleton with feedback the atrophy can be prevented from occurring in the first place. FIG. 12 illustrates another application of the sensory feedback processing unit 106, used along with a hand exoskeleton in administering physiotherapy to prevent muscle atrophy. In order to diagnose, a wearable device like an exoskeleton with inbuilt sensors 1202 is worn on the body part 1204 of a patient. The sensor array includes a plurality of sensors arranged on the exoskeleton 1202 to sense the set of parameters associated with muscle health of the body part 1204. The sensors scan the body part 1204 and send the information to the sensory feedback processing unit 106 as well as the output devices 1206 for the healthcare professional to monitor any improvement in muscle health. The sensory feedback processing unit 106 processes the information and provides visual and/or audio feedback on the condition of the muscle to a healthcare professional on one or more output devices 1206. In order to administer the therapy, the healthcare professional analyses the information displayed on the output devices 1206 and sends a direction to the sensory feedback processing unit 106. The sensory feedback processing unit 106 provides tactile simulations along with necessary actuations through the one or more actuators of the exoskeleton to the body part 1204 of the patient. The tactile simulations may include, but not limited to, heat, texture, pressure, vibro-tactile, electro-tactile, or mechano-tactile, temperature, pressure simulations along with the exo-skeleton movement. Further, in order to vary the therapy, the healthcare professional can regulate various means like frequency, amplitude, etc., based on the readings observed from output devices 1206.

In various embodiments of the present disclosure, the feedback system may be integrated into the main system or be connected as an add-on system. Also the input and outputs from main system to the input and outputs to the user maybe transmitted in a wired or a wireless manner.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A sensory feedback system (101), comprising:
a sensor array (104) arranged with respect to an entity (102) to sense a set of parameters associated with an operation of the entity (102); and
a sensory feedback processing unit (106) configured to generate sensory feedback based on the sensed parameters, and provide the sensory feedback to an operator of the entity (102) by way of at least one of: an actuator and an output device, thereby enabling control of the operation of the entity (102), wherein the sensory feedback is at least one of: tactile feedback, visual feedback, and audio feedback.

2. The sensory feedback system (101) of claim 1, wherein:
the entity is a vehicle (402) and the operation of the entity includes navigation along a route,
the sensor array (404) includes a plurality of proximity sensors arranged with respect to the vehicle in one or more orientations of the vehicle (402) to cover one or more blind spots of the vehicle (402),
the set of parameters include at least one of: a distance between the vehicle (402) and one or more obstacles in the one or more orientations, and a shape of the one or more obstacles,
the sensory feedback processing unit (106) provides the sensory feedback to an operator of the vehicle (402) in respect to the control of the navigation of the vehicle (402), through at least one of: a seat of the operator, a steering mechanism of the vehicle, a control pedal of the vehicle, and a display device of the vehicle, and
the tactile feedback is at least one of: heat, pressure, displacement, vibration, electrical, and texture based simulation.

3. The sensory feedback system (101) of claim 1, wherein:
the entity is a movable arm of an equipment (500, 600) having a pivoting element and the operation of the entity includes handling of the entity,
the sensor array includes a plurality of motor current sensors arranged to sense a torque at a pivoting point (502, 504, 602, 612) of the pivoting element,
the set of parameters include a force applied by the movable arm,
the sensory feedback processing unit (106) provides the sensory feedback to an operator of the equipment (500, 600) in respect to the control of the entity and the load carried by the entity, through at least one of: a seat of the operator, a steering mechanism of the equipment, a remote control device of the equipment, and a display device of the equipment, and
the tactile feedback is at least one of: heat, pressure, displacement, vibration, electrical, and texture based simulation.

4. The sensory feedback system (101) of claim 3, wherein the equipment (500, 600) is one of: an industrial vehicle, an industrial robot, a medical equipment, a prosthetic device, and a bomb defusal equipment.

5. The sensory feedback system (101) of claim 1, wherein:
the entity is a movable arm of an equipment (500, 600) having a gripping element and the operation of the entity includes handling load carried by the entity,
the sensor array includes a plurality of sensors arranged to sense imbalance or tilt at a gripping point (506, 508, 604, 606, 608, 610) of the gripping element,
each sensor is at least one of: a proximity sensor and a pressure sensor,
the set of parameters include a back pressure at the movable arm and an orientation of load with respect to the movable arm,
the sensory feedback processing unit (106) provides the sensory feedback to an operator of the equipment (500, 600) in respect to the control of the load held by the entity, through at least one of: a seat of the operator, a steering mechanism of the equipment, a remote control device of the equipment, and a display device of the equipment, and
the tactile feedback is at least one of: heat, pressure, displacement, vibration, electrical, and texture based simulation.

6. The sensory feedback system (101) of claim 5, wherein the equipment (500, 600) is one of: an industrial vehicle, an industrial robot, a medical equipment, a prosthetic device, and a bomb defusal equipment.

7. The sensory feedback system (101) of claim 1, wherein:
the entity is an unmanned aerial vehicle (902) and the operation of the entity includes handling orientation and navigation of the entity,
the sensor array includes a plurality of sensors arranged with respect to the unmanned aerial vehicle (902) in one or more orientations of the unmanned aerial vehicle (902) to cover one or more blind spots of the unmanned aerial vehicle (902), and maintain orientation of the unmanned aerial vehicle,
each sensor is at least one of: a proximity sensor and an orientation sensor,
the set of parameters includes at least one: of a proximity of one or more obstacles with respect to the entity, and pitch, yaw, and roll of the entity,
the sensory feedback processing unit (106) provides the sensory feedback to an operator (904) of the equipment in respect to the control of the orientation and navigation of the entity, through a remote control device of the unmanned aerial vehicle, and
the tactile feedback is at least one of: heat, pressure, displacement, vibration, electrical, and texture based simulation.

8. The sensory feedback system (101) of claim 1, wherein:
the entity is one or more wearable bands, and the operation of the entity includes assistive guidance to a person having visual impairment,
the sensor array (1002, 1004) includes a plurality of proximity sensors arranged on the one or more wearable bands to sense the set of parameters associated with assistive guidance to the person regarding obstructions in front of the person,
the set of parameters includes at least one of: a distance between the person and one or more obstacles, and a shape of the one or more obstacles,
the sensory feedback processing unit (106) provides the tactile feedback to the person in respect to the control of assistive guidance provided to the person, through the one or more wearable bands, and
the tactile feedback is at least one of: heat, pressure, displacement, vibration, electrical, and texture based simulation.

9. The sensory feedback system (101) of claim 1, wherein:
the entity is a wearable spectacle, and the operation of the entity includes assistive guidance to a person having at least one of: visual impairment and hearing impairment,
the sensor array (1008) includes a plurality of proximity sensors arranged on the wearable spectacle to sense the set of parameters associated with assistive guidance to the person regarding obstructions in front or side of the person,
the set of parameters includes at least one of: a distance between the person and one or more obstacles and a shape of the one or more obstacles,
the sensory feedback processing unit (106) provides the tactile feedback to the person in respect to the control of assistive guidance provided to the person, through one or more temple tips of the wearable spectacles, and
the tactile feedback is at least one of: heat, pressure, displacement, vibration, electrical, and texture based simulation.

10. The sensory feedback system (101) of claim 1, wherein:
the entity is one of: a wearable band (1102) and an exoskeleton (1202) worn on a body part (1104, 1204) of a patient, and the operation of the entity includes monitoring of muscle health of the body part,
the sensor array includes a plurality of sensors arranged on one of: the wearable band (1102) and the exoskeleton (1202) to sense the set of parameters associated with muscle health of the body part (1104, 1204),
the sensory feedback processing unit (106) provides the tactile feedback to the patient in respect to the monitoring of muscle health of the body part and the visual and/or audio feedback to a healthcare professional monitoring the patient, through one or more actuators of one of: the wearable band (1102) and the exoskeleton (1202), and
the tactile feedback is at least one of: heat, temperature, pressure, texture, vibro-tactile, electro-tactile, and mechano-tactile based simulation.
